(19) United States
(12) Patent Application Publication
(10) Pub. No.: US 2006/0276702 A1
(43) Pub. Date: Dec. 7, 2006

(54) Neurophysiological wireless bio-sensor

(76) Inventor: William McGinnis, Cincinnati, OH (US)

Correspondence Address: LADAS & PARRY LLP, 224 SOUTH MICHIGAN AVENUE, SUITE 1600, CHICAGO, IL, 60604, US

(21) Appl. No.: 11/292,861

(22) Filed: Dec. 2, 2005

Related U.S. Application Data
(63) Continuation-in-part of application No. 11/144,214, filed on Jun. 3, 2005.

(51) Int. Cl. A61B 5/04

(52) U.S. Cl. 600/372

(57) ABSTRACT

This invention is directed to a wireless bio-sensor electrode for recording bio-potentials elicited from a subject or for providing a stimulus to a subject. A preferred embodiment is a wireless bio-sensor electrode for eliciting from a subject bio-potentials including averaged evoked potentials, nerve conduction studies, electromyographic activity, electrocardiogram or electroencephalogram, or for providing a stimulus to the subject for eliciting said bio-potentials. Another embodiment is the use of the wireless bio-sensor electrode for recording far-field and near-field bio-potentials in a subject in real-time and in a real-time neurophysiological monitoring/testing system.

### BACKGROUND OF INVENTION

This is a continuation-in-part of pending U.S. Ser. No. 11/244,214, filed on Jun. 3, 2005, and entitled Method Of Using Dermatomal Somatosensory Evoked Potentials In Real-Time For Surgical And Clinical Management which is hereby incorporated by reference.

### FIELD OF THE INVENTION

This invention relates generally to the field of devices and systems for neurophysiological monitoring/testing/assessment in both clinical and intraoperative settings.

Elicitation and recording of electrophysiological potentials via electrodes on predetermined sites on the body, such as electrocardiograms (ECG), electromyographic activity (EMG), and evoked potentials such as somatosensory evoked potentials (SSEP) and dermatomal somatosensory evoked potentials (DSSEP), are all well documented in the medical literature. Somatosensory evoked potentials are assessed neurophysiologically for latency and amplitude measurements that reflect mixed nerve (both sensory and motor fiber) function (SSEP) and nerve root function (DSSEP). Generally, mixed nerve SSEPs are robust and easily obtained from peripheral stimulation sites, and their use is well established clinically for evaluating the electrophysiological presentation in patients with neurological symptoms. Anatomically innervated by multiple overlapping nerve roots, SSEPs cannot be used specifically to identify problems found with individual nerve roots. DSSEPs are used to assess individual nerve root function.

When a patient undergoes a test of the functional presentation of their nervous system, it is common practice to assess nerve function by recording with electrodes the electrophysiological activity present in a muscle innervated by the nerve, or to stimulate the surface of the skin near the nerve or in a distribution of the nerve with an electrical current and record the current transported along the pathway of the nerve to the spinal cord. The current transported by the nerve to the spinal cord ultimately reaches the location in the brain where cortical control of the nerve is located. If recording electrodes are placed over the spinal cord or over the area of the brain where cortical control of the nerve is located, bio-potential amplifiers will record a signal when the signal reaches the recording electrode. Generally, an averaged sample is taken of the time the signal takes to reach the electrode, marked as the latency, or the time the stimulus takes to reach the recording electrode. Equipment for obtaining such electrophysiological measurements generally requires manual marking of latency, requiring the practitioner to correlate the measurement and assess the neurological correlation of the finding, a process that can be time-consuming and technically demanding.

Although obtaining DSSEPs is non-invasive, and relatively inexpensive, it is technically demanding, and reproducible results are difficult to obtain. The literature identifies the primary recording site for a dermatomal response as being over the somatosensory cortex. However, signals from the cortex are known to be ambiguous at best, in both awake and in anaesthetized patients. Owen et al, (Spine vol. 18, No. 6, pgs 748-754 (1993)) in studying the differences in the levels of the DSSEP and nerve root involvement, report variable results in the peripheral innervations patterns of the dorsal nerve roots in the cervical and lumbar spine. U.S. Pat. No. 5,338,587 addressed the lack of reproducibility of responses detected at the cerebral cortex through static comparisons of transport times (latency) of signals from different stimulating electrodes.

It has been surprisingly found that superior and robust DSSEP waveforms may be obtained at a subcortical recording site. Reproducible high-confidence DSSEP data would be a considerable advance.

Furthermore, a software for evaluating collected electroneurophysiological data, validating quality collection, confirming stimulus-recording placement, comparing collected samples to normal based on neurological correlation and providing a comprehensive neurophysiological assessment based on the collected electrophysiological data, would be a significant advance over current practice. More advantageous still to clinicians and surgeons would be to be able to compare elicited evoked potentials in real-time by performing comparisons between waveform data and assessing the changes in real-time. Capturing such critical physiological data in real-time has never before been achieved. Real-time feedback and assessment of elicited waveform data would be useful to a practitioner or a surgeon in helping prevent the likelihood of nerve damage during a procedure, particularly intraoperatively.

Numerous problems are associated with conventional methods of electrode placement. The vast preponderance of recording requires stimulation and recording montages that require multiple electrodes being applied to a single subject, often providing an opportunity for confusion, non-sequential solicitation and protocol breech of electrophysiological data. In a clinical setting, the clinician has visual appreciation of electrode placement and site confirmation, but with as many as eight paired electrodes, sixteen total electrodes on a single side, logistical coordination is a challenge. Further, in the operative suite where multiple agenda's are being implemented, and as many as sixty to seventy electrodes are applied, logistical coordination can be a major issue.

The prior art teaches a wireless electrode having the capability for electrical and neuromuscular stimulation of a subject (for example, U.S. Published Patent Application Nos. 20040173220, 20050182457, 20020010499), heart-rate and somatic monitoring (for example, U.S. Published Patent Application Nos. 20050116820, 20050113661, 20050038328). U.S. Published Patent Application No. 20040015096 discloses a wireless, remotely programmable electrode transceiver assembly that sends electromyographic activity (EMG) signals via wireless transmission to a base unit. The base unit obtains a patient's EMG signal from the wireless transceiver and supplies the signal to a monitor unit for display. U.S. Published Patent Application Nos. 20040015096 and 20030109905 teach wireless surface electrodes that record spontaneous EMG activity, digitalize, encode, and then transmit over radio frequency (RF) to a receiver, having two-way communication between the electrodes and data receiver, which has application in biofeedback and neuromuscular disorders.

The prior art does not teach a wireless bio-sensor electrode that can record a physiological signal occurring in time between a pair of electrodes, generating a signal time-locked to a given stimulus, the generated signal being amplified by a differential amplifier, the signal being processed at the site of the recording and then transmitted to a remote recorder. Those skilled in the art will appreciate that such capabilities would be of certain use during a wide variety of clinical, and particularly intraoperative, procedures.

### SUMMARY OF THE INVENTION

In one aspect of this invention, a wireless bipolar bio-sensor is provided for attaching to the body of a subject for recording a biopotential signal elicited from the subject and reflecting a neurological function, the bio-sensor comprising: a pair of electrodes capable of recording a signal from the subject; a differential amplifier in contact with the electrodes and capable of generating an amplified differential signal from signal recorded between the electrodes; a miniaturized system-on-a-chip (SOC) attachment in contact with the differential amplifier configured to process the signal received from the amplifier; and an infra red light transmitter/receiver connected to the SOC attachment and capable of receiving optical power from a remote ir-light source transceiver, and of transmitting the signal thereto. The bio-sensor is optically powered by a remote ir-light source transceiver being capable of transmitting optical power to the sensor and receiving a signal therefrom.

In one embodiment, the electrodes are discs made of silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof.

In another aspect of the invention, the bio-sensor records a signal measuring the subject's spontaneous activity. In a preferred embodiment, the the signal is an electromyographic signal, electrocardiographic signal or an electroencephalographic signal.

In another aspect of the invention, the signal is a measurement of the subject's response to a pathology experienced by the subject, including a trauma, a circulatory change, a degenerative change, a metabolic change, an infection, a chemical insult, radiation, or a neoplastic change. In a preferred embodiment, the pathology is the result of a surgical intervention.

In yet another aspect, the bio-sensor measures a signal evoked from the subject in response to an applied stimulus. In a highly preferred embodiment, the response is time-locked to the stimulus. Such signals may be a somatosensory evoked potential, a dermatomal somatosensory evoked potential, a motor evoked potential or a nerve conduction potential. The applied stimulus may be electrical, sonar, mechanical, tactile or optical.

In a highly preferred embodiment, the signal results from a change in the subject's response to the applied signal as a result of a pathology experienced by the subject. The pathology may be a result of a trauma, a circulatory change, a degenerative change, a metabolic change, an infection, a chemical insult, radiation, or a neoplastic change. In a highly preferred embodiment, the pathology is the result of a surgical intervention.

In a particular embodiment of the bio-sensor, the SOC attachment is configured to integrate the following: signal acquisition; filtering the signal; averaging the signal; summating the averaged signal; converting the signal to a digital signal; signal conditioning to assign a digital latency value; and transmitting the digital signal to a remote receiver.

In another embodiment of the bio-sensor, the pair of electrodes is housed in a first layer having on its distal surface an adhesive area for cutaneous or percutaneous conductive attachment to the subject's musculature, the pair of electrodes being transferred to an electrode substrate material proximally in contact with a second unexposed layer comprising the differential amplifier, the SOC attachment and the infra red light transmitter/receiver, the second layer being covered by a third exposed layer comprising an insulating material and extending to the circumferential borders of the first layer, the third layer having a transparent portion for transmitting and receiving power.

In yet another embodiment, the electrodes are silver-silver chloride.

In a further embodiment, the electrodes are needle electrodes. In a preferred embodiment, the needle electrodes are in-housed percutaneous needles for percutaneous attachment to the subject's musculature, and the proximal ends of the needle may be attached to the SOC attachment and embedded in an electrode substrate material. In one embodiment, the bio-sensor allows for adaptation of percutaneous needles. In another embodiment, the SOC attachment is configured to integrate the following: signal acquisition; filtering the signal; averaging the signal; summating the averaged signal; converting the signal to a digital signal; signal conditioning to assign a digital latency value; and transmitting the digital signal to a remote receiver. In a further embodiment, the electrodes are silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof. In a highly preferred embodiment, the electrodes are gold.

In yet another aspect of the invention, a bio-sensor is provided wirelessly powered for transmission of an electrical stimulus to a subject, comprising: a pair of electrodes providing for delivery of an electrical stimulus to the subject's skin; and a SOC attachment in contact with the electrodes, and including: a stimulus circuit providing transcutaneous stimulation to the subject via the electrodes; a receiver means for activating a constant current stimulator to deliver a stimulus; a means for controlling the duration and intensity of the stimulus; and an infra red light transmitter/receiver means connected to the SOC attachment and capable of receiving optical power from a remote ir-light source transceiver, and of transmitting a feedback signal thereto. The bio-sensor is optically powered by a remote transceiver connected via a USB port to a computer. In one embodiment, the stimulation is provided in software-controlled intensities. In a further embodiment, the stimulation is provided in intensities of between about 0.5 mA and 10 mA. In yet another embodiment, the electrodes are discs of silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof. In a preferred embodiment, the electrodes are silver-silver chloride. In a further embodiment, the pair of electrodes is housed in a first layer having on its distal surface an adhesive area for cutaneous or percutaneous conductive attachment to the subject's musculature, the pair of electrodes being transferred to an electrode substrate material proximally in contact with a second unexposed layer comprising the differential amplifier, the SOC attachment and the infra red light transmitter/receiver, the second layer being covered by a third exposed layer comprising an insulating material and extending to the circumferential borders of the first layer, the third layer having a transparent portion for transmitting and receiving power. In yet another embodiment, the distal surface is a stimulating surface.

Systems and methods for neurophysiological measuring/monitoring/testing are also provided comprising the biosensors of the invention, a transceiver station comprising ir-transmitters/receivers means for powering, and for data reception from, the one or a plurality of the bio-sensor, the transceiver being powered by a computer via a USB port; and software enabling a computer, the software comprising interacting with the bio-sensors and the transceiver station, reading the data from the USB port, displaying and assessing the data. In preferred embodiments of these systems and methods, the software further comprises directing serial collection of signal data and real-time display, comparison and assessment of the collected signal data. In preferred embodiments, software is provided to generate a deviation from normal warning signal via a visual, audible or electronic means. In other preferred embodiments, software is provided for providing and displaying an icon on a computer screen responsive to a command by a computer user, wherein the icon appears on the screen and prompts a user to select an option consisting of take a patient history, select a recording protocol, confirm proper electrode placement, input parameters, record a sequence, analyze data, archive data, or generate a report. Yet other preferred embodiments further comprise an apparel for the subject to wear, having apertures for guiding placement of the apertures in the stocking correlating with a specific electrode montage.

In another preferred embodiment is provided a computer data signal embodied in a carrier wave by a computing system and encoding a computer program for executing a computer process comprising instructions for executing real-time comparison and assessment of evoked potentials, nerve conduction studies, electromyographic activity, or electrocardiographic or electroencephalographic signals.

### BRIEF DESCRIPTION OF THE DRAWINGS

FIG. 1 is a view of one construction of the bio-sensor electrode.

FIG. 2 illustrates an embodiment of the wireless bio-sensor having an adaptation for EMG needle electrodes.

FIG. 3 illustrates an embodiment of the infrared (ir) light source transceiver station system.

FIG. 4 represents a wireless medical neurophysiological monitoring/testing set-up.

FIG. 5 is a schematic representing one configuration of a bio-sensor averaging electrode.

FIG. 6 is a schematic representing one configuration of a bio-sensor stimulating electrode.

FIG. 7 is a schematic representing one configuration of a bio-sensor electromyographic activity electrode.

FIG. 8 shows a diagram of the instrumentation differential OpAmp amplifier.

The same reference numerals have been used, where possible, to designate the same elements that are common to the figures.

The following terms used in the specification are defined as follows:

Evoked potentials (EP): a change in the electrical activity of the nervous system in response to an external stimulus. Stimuli are applied to specific motor or sensory receptors and the resulting waveforms are recorded along their anatomic pathways in the peripheral and central nervous system. Somatosensory evoked potentials (SSEP): changes in the electrical activity manifested as waveforms elicited by stimulation of specific peripheral sensory nerves and recorded from peripheral and central nervous system structures. An SSEP waveform is generally a complex waveform with several components specified by polarity and average peak latency. The polarity and latency depend upon subject variables such as age and gender, stimulus characteristics such as intensity and rate of stimulation, and recording parameters, such as amplifier time constants, electrode placements and electrode combinations. Dermatomal somatosensory evoked potentials (DSSEP) are waveforms generally recorded at the scalp generated from repeated stimulation of a specific dermatome.

Spontaneous electromyographic activity (sEMG): recording and study of spontaneous activity of a muscle with a recording electrode (either a needle electrode for invasive EMG or a surface electrode for kinesiologic studies). Point-surface electromyographic activity (EMG) is very poor reflector of muscle activity, even with efficient filtering of artifact. EMG is a low amplitude, fast-frequency signal, and transmission of the signal using radio frequency can skew or contaminate the physiological signal with unwanted radio frequencies occurring in the spectrum. Needle recording from the body of the muscle is generally regarded as superior, being uncontaminated with artifact through highly resistant skin layers.

Compound muscle action potential (CMAP): summation of nearly synchronous muscle fiber action potentials recorded from a muscle, produced by stimulation of the nerve supplying the muscle either directly or indirectly.

Motor (neurogenic) evoked potential (MEP): a compound muscle action potential produced by either transcranial magnetic stimulation or transcranial electrical stimulation.

Nerve conduction studies (NCS): the speed of conduction of an action potential along the nerve.

Nerve action potential (NAP): an action potential recorded from a single nerve.

Electrocardiograph (ECG): measurement of rate and regularity of heartbeats, and size and position of the chambers of the heart, and presence of any damage to the heart.

Electroencephalograph (EEG): measurement to detect abnormalities in the electrical activity of the brain.

### DETAILED DESCRIPTION

A signal may be recorded from a subject reflecting spontaneous biological activity in the subject, such as electromyographic activity, electrocardiographic activity or encephalographic activity. This activity may be altered by the subject's response to pathology, for example when a surgeon damages a nerve during an operative procedure, or as a result of change in circulation, amongst other pathologic conditions.

An evoked potential may be recorded from a subject in response to an applied stimulus, where the applied stimulus is electrical, via a stimulating electrode, as in procedures to obtain somatosensory evoked potentials, dermatomal somatosensory evoked potentials, or motor evoked potentials, or where the applied stimulus is optical as in procedures to obtain visual evoked potentials, sonar as in procedures to obtain brain stem evoked potentials, or mechanical as in pedicle screw procedures or nerve conduction studies. This evoked potential response may be altered by the subject's response to a pathology such as, for example, trauma resulting from the surgeon's knife, degenerative changes, circulatory changes, metabolic changes, infection, chemical changes, radiation, or neoplastic changes. The recorded signal may be time-locked to the stimulus to produce a more robust recording by producing an averaged response with reduction in background noise.

Generally, these activities are measured via conventional wire electrodes.

In this invention a wireless neural bio-sensor is provided having an integrated system-on-a-chip (SOC) technology and data acquisition/transmission that has achieved the elusive balance of low-noise, low-power signal processing and wireless data communication.

The bio-sensor is attached to a subject undergoing a neurological procedure, such as measuring/monitoring/testing of averaged evoked potentials, nerve conduction studies, electromyographic activity, compound muscle action potentials, neurogenic evoked potentials, electrocardiogram or electroencephalogram. Signals that can be measured by the bio-sensor can be signals in response to stimulus that is electrical, physiological, biological, metabolic, viral or mechanical, and particularly in response to a mechanical insult during a surgical procedure.

The wireless bio-sensor electrode is a self-contained single channel biopolar device comprising a pair of electrodes for recording a signal between the electrodes time-locked to delivery of a stimulus, a differential amplifier receiving the input from the pair of electrodes, a miniaturized system-on-a-chip (SOC) for processing the signal, and a receiver/transmitter means for receiving power wirelessly, for receiving or transmitting data wirelessly, and for interacting with a digital transmitter of stored electrical data. The bio-sensor is powered by light, using optical near-infrared light for powering and transmission. Signals recorded are processed at the site of the recording, and processed signal data is wirelessly transmitted to a remote receiver via an optically powered near-infrared light transmitter, thereby reducing mechanical and electrical artifacts.

The electrodes in contact with the subject's skin establish two electrical poles that provide the physical boundaries for detecting near and far electrical fields, under which physiological electrical activity is occurring. The independent fields reflect changing patterns in each electrode, the input from the electrodes being assigned a designated polarity positive or negative by convention. The differential op-amp then propagates the signal that is inverted/non-inverted between the two electrodes. Like-signals at each electrode are regarded as nonevents. A depolarization/repolarization as a function of time is a significant electrophysiological event. If a significant event has occurred in the electrical fields as a function of some given stimulus, the electrical event will have distribution to it. The neural structures in the field will depolarize, then as a time course will repolarize. If the signal depolarizes at one electrode, then that electrode will change its electrical properties, and as a time course a different change should take place at the other electrode. Only changes that are different are regarded as significant electrical events.

The integral design of our differential op-amp allows for identification and removal of DC biased potentials at the site of our electrodes. Its functionality provides linearity to inverting and non-inverting polarities and integrates elusive low power requirements. Propagating signal across resistors in series permits significant application of voltage to a biosignal embedded in a hostile electrical background that generates a superior neurophysiological representation.

The SOC is an integrated circuit (IC) designed in complementary metal oxide semiconductor (CMOS) technology. The receiver means in the bio-sensor comprises a light collector that is visible at is proximal end. When light is detected the IC converts light to current to power the sensor. The light collector is an attached to a photodiode which converts light to current. The current is then applied to all of the electronic components. All the electrical components are chips embedded in a substrate material. The chips attached to each other and to the electrodes with metal oxide connections. The transmitter receives the processed data from the output chip and turns on the LED light emitting diode. The LED then send the data to the base station. The light collector, converter, processing chips, electrodes, and data transmitter are connected in IC.

Recording Averaging Bio-Sensor Electrode

In one preferred embodiment is provided a recording averaging bio-sensor electrode having a SOC that is capable of integrating the following: filtering the bandwidth of the amplified recorded signal; averaging the signal time-locked to stimulus; summating the averaged signal; converting the summated averaged signal from analog to digital; conditioning the signal to assign a digital latency value: transmitting the digital signal to a remote recorder via a light-emitting diode (LED), the sensor being powered via the LED by a near infrared light transmitter photodiode source.

More particularly, the pair of electrodes is housed in a first layer having on its distal surface an adhesive area for cutaneous conductive attachment to a subject's skin, the pair of electrodes being transferred to an electrode substrate material proximally in contact with a second unexposed layer connected to the SOC attachment, the second layer being covered by a third exposed layer comprising an insulating material and extending to the circumferential borders of the first layer, the third layer having a transparent portion for transmitting signal data and receiving power from a remote power source. The SOC-containing platform further comprises electronics for a light emitting diode (LED) for providing power and signal reception/transmission.

Although the SOC chip could be a CMOS (complimentary metal-oxide semiconductor) chip, the approach is not intended to be limited to any particular chip technology, it being understood that there are several chip technologies capable of supplying the above capabilities.

The electrodes are silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof. In an ideal embodiment of the bio-sensor, the electrodes are discs of silver-silver chloride.

EMG Needle Bio-Sensor Electrode

In another embodiment, a free run needle electromyographic activity (EMG) bio-sensor is provided for percutaneous conductive attachment to a subject's musculature, for recording and evaluating muscle innervation. In this bio-sensor, the electronics for signal averaging are abated and the electrode comprises two needles that are manually inserted into the musculature by pressing the lateral insertion tabs on the sensor. In a preferred embodiment, the needles are gold needles of 13 mm/27 gauge. The bio-sensor in-houses a pair of percutaneous needles, held above surface contact within an expandable plastic dilator, and wherein when force is applied to the proximal end of the needle, expansion allows for the needle to be percutaneously positioned in the subject's musculature. In one embodiment of the bio-sensor, the third layer of the bio-sensor allows for adaptation of the percutaneous needles.

Stimulus Bio-Sensor Electrode

In another aspect of the invention, a wireless bio-sensor is provided for providing transcutaneous constant current stimulation of a subject, and providing control of duration and intensity of the stimulus inside the bio-sensor, through a photodiode optically powered near infrared light transmitter. The SOC attachment of this bio-sensor comprises a receiver means for activating a constant current stimulator to deliver a stimulus, and a means for controlling the duration and intensity of the stimulus, wherein the duration and intensity is controlled at the site of the stimulation.

In the stimulating electrode configuration, the wireless bio-stimulation electrode has first layer having an adhesive strip on its distal side for placement against the skin of the subject, and housing a pair of stimulating electrodes, comprising a metal such as silver chloride, silver-silver chloride, gold or tin (preferably 8 mm-gold-plated, Ag, Ag/Ag—Cl disc) with positive and negative orientation for providing bi-phasic surface stimulation, the electrodes being attached proximally to a platform containing electronics for a constant current stimulator and micro processing controls, as well as, a second platform that contains electronics for a light emitting diode (LED) for providing power and data reception and control of duration and intensity of the stimulus from remote firmware.

Bio-Sensor System

It will be evident to those skilled in the art that the use of such bio-sensors and bio-sensor systems would be contemplated in neurophysiological monitoring and testing settings, and particularly in real-time neurophysiological monitoring and testing. Accordingly, also provided is a bio-sensor recording system. The bio-sensor recording system contemplates the use of a plurality of such bio-sensors having wireless interface with firmware, either for recording signals from different recording sites on the subject, or for providing stimulation to the subject at different sites on the subject, or both.

In another aspect therefore, a system is provided, comprising one or a plurality of the bio-sensor, in which the firmware with which the wireless electrode interacts comprises a unit housing an infra-red light source with USB interface to a standard computer for power and control, running software that provides pattern recognition of the light source unit and looks for and queries any signal from the bio-sensor creating displays and assessment. To facilitate recognition among bio-sensors, the light source unit uses a photo-filtering labeling technology.

In a preferred mode, the data is transmitted to the remote receiver in real-time. In another aspect, the bio-sensor electrode is used in conjunction with electrode placement apparel, such as a stocking or sleeve worn on the subject's lower or upper limb or trunk portion, and having apertures corresponding to a particular electrode montage for guiding placement of the electrodes.

In a further aspect, the invention provides a computer data signal embodied in a carrier wave by a computing system and encoding a computer program for executing the computer processes driving the bio-sensor system, the program comprising instructions for executing measurement monitoring/testing of neural signals, particularly in real-time.

Those skilled in the art will appreciate that such capabilities would provide vital and critical help to a surgeon or a practitioner during a wide variety of procedures, in clinical, and particularly, in intraoperative procedures.

Various figures show different aspects of the system, and, where appropriate, reference numerals illustrating like components in different figures are labeled similarly. It is understood that various combinations of components other than those specifically shown are contemplated. Further, separate components are at times described with reference to a particular system embodiment, and while such description is accurate, it is understood that these components, with the variants described, are independently significant and have patentable features that are described separate and apart from the system in which they are described.

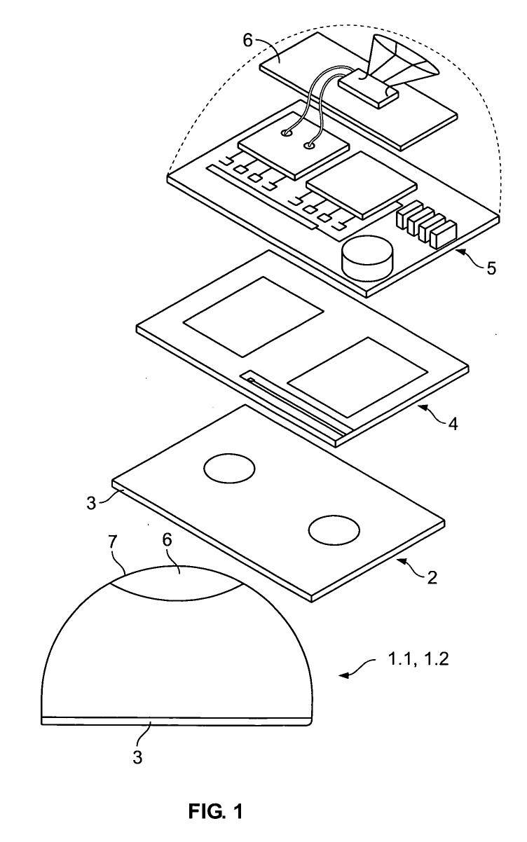

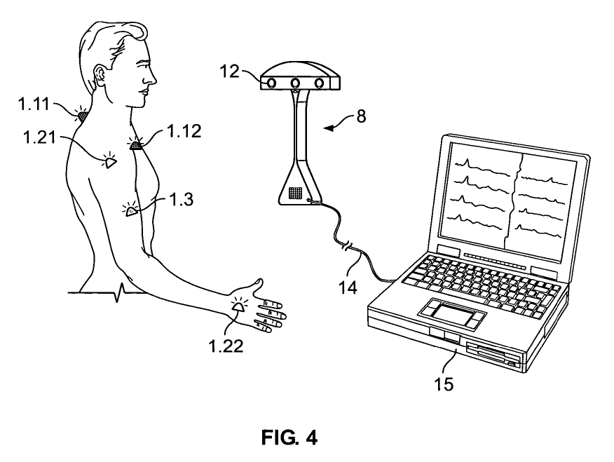

FIG. 1 is a view of the construction of one embodiment of the bio-sensor electrode (1) for recording far-field and near-field bio-potentials elicited from a subject, and providing surface stimulation. Lower conducting metal base platform (2) houses a pair of disc electrodes, each electrode being single channel electrodes with a dual interface, two inputs and two outputs, so that in a single sensor can record from a site or stimulate a site on the subject. Each bio-sensor can thereby be either a recording or a stimulating bio-sensor electrode. Platform (2) has situated on its distal surface adhesive layer (3) for attachment to the skin of a subject. The disc electrodes on platform (2) are made of any high resistance conducting metal that has low impedance, such as for example, but not limited to, silver chloride, silver-silver chloride, gold and tin. (2) is attached proximally to distal portion of (4), comprising the electrodes transferred to an electrode substrate material. (4) is attached proximally to platform (5), comprising a differential amplifier in contact with a system-on-a-hip (SOC) attachment, the SOC including the processing of an elicited signal including amplifying, filtering, averaging, summating, digitally converting and transmitting the signal to a computer for display/assessment. The SOC attachment on (5) comprising the required processing for activating a constant current stimulator to deliver a stimulus, and for controlling the duration and intensity of the stimulus. Transparent light collector (7) atop the bio-sensor in the outer covering of the bio-sensor provides for signal reception/transmission. The bio-sensor is powered by a near ir-light source transceiver station from which an ir-modulated light beam is directed toward transparent light collector (7). Remote photodiode light source transceiver station (8) is shown in FIGS. 3 and 4. Any miniaturized power source (including, but not limited to, pizer, chemical, battery, and LED) will serve, but when choosing a miniaturized power source, those skilled in the art will appreciate that a light emitting diode power source overcomes the drawbacks of battery power source shelf-life.

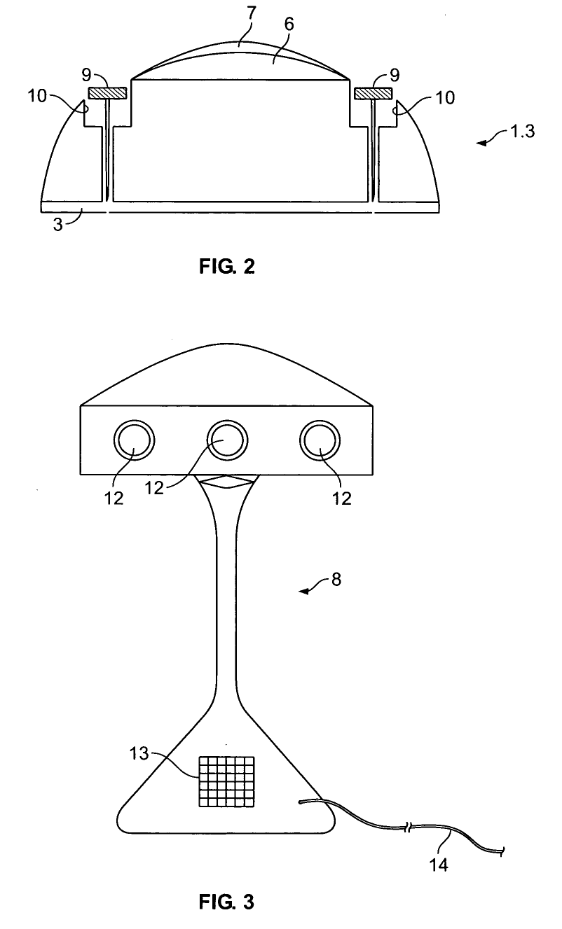

FIG. 2 depicts another embodiment of the bio-sensor being a percutaneous bio-sensor adapted for EMG recording, and in which the pair of electrodes is a pair of needle electrodes (9) for percutaneous attachment via sunk portions (10) when tapped down into the musculature of the subject. When the needles are tapped down in sunk portions (10), they protrude through the base metal portion (3) of the bio-sensor through the skin and into the musculature of the subject.

FIG. 3 illustrates one embodiment of the bio-sensor's photodiode light source (8), being an infra red (ir) transmitter receiver where (12) represents infra red light emitters housed inside a movable dome with an adjustable base for changing the angle of direction for being aimed in the direction of the subject, and sourced at circuitry comprising the internal electronics (13) and powered via USB port (14).

FIG. 4 illustrates the approach in a wireless medical neurophysiological monitoring/testing set-up in which wireless electrodes are communicating wirelessly with the transceiver station which in turn is in communication via a USB cable with a computer. The computer contemplated in a system such as those described herein is not limited to a personal or desktop or mainframe computer, but could include a hand-held device such as a Palm™ device. Numbers represented in previous drawings are the same as in the previous figures. In this figure, recording averaging bio-sensors and stimulating bio-sensors are shown attached to the subject. Averaging recording bio-sensors (1.11) and (1.12) are placed to record, respectively, over the posterior cervical spine and the brachial plexus. Stimulus-delivering bio-sensors (1.21) and (1.22) are placed, respectively, to deliver a stimulus to the C5 dermatome and to the C6 dermatome. Recording EMG bio-sensor (1.3) is placed to record over the bicep. Light is received from, and signals transmitted to, infra red (ir) transmitter receiver (8). Signals received by (8) are passed via USB interface (14) to computer (15) for real-time digital display and assessment by software run by the computer.

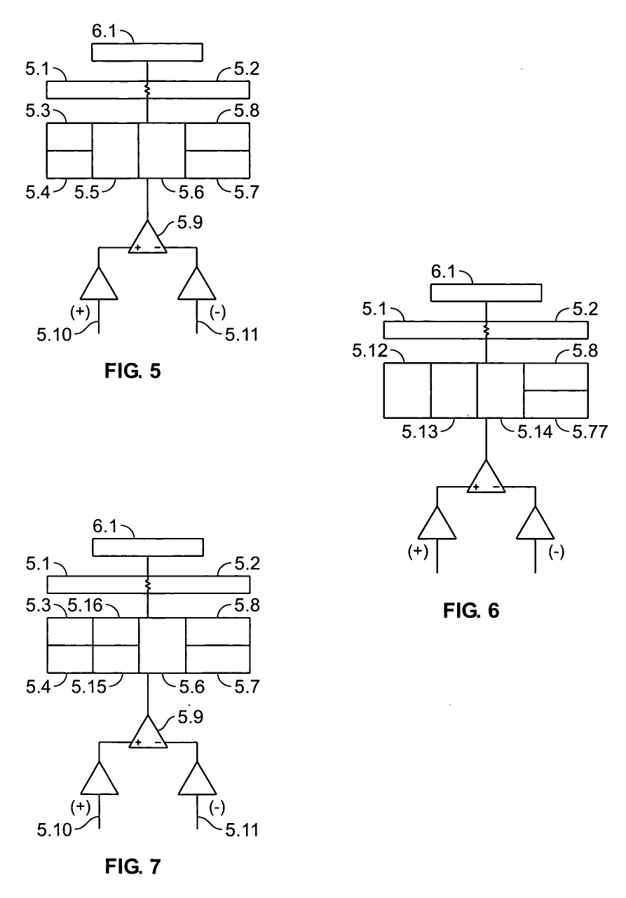

FIGS. 5-7 represent embodiments of the electronics of the bio-sensors.

FIG. 5 shows a schema of the electronics for the processing via amplifiers/capacitors/resisters (5.7) by microcontroller (5.8). Some of the numbers referred to in FIG. 5 represent numbers from previous FIGS. 1, 2, 3 and 4. In the schema of FIG. 5, a signal is emitted via input (5.10) and output (5.11), and passes through differential operational amplifier (OpAmp), (5.9). At (5.3) the band width is filtered to eliminate unwanted slow or fast frequencies that are not in the physiological spectrum. For example, for upper extremities, the recording window is approximately 50 msec. When a C6 dermatome is stimulated, it is known that the physiological response will be approximately 28 msec, and slow and fast frequencies not falling in that range are filtered to improve the signal to noise ratio. Successive trials are made and successive processed signals are summated and averaged (5.5) to give the summated averaged potential which is then converted from analog to digital (5.6) by an A-D converter. LED (6) converts light to power at (5.1). Then the digital signal is transmitted at (6.1) to infra red light source (1) which passes the signal via USB interface (14) to computer (15) having software for real-time for digital display and assessment.

FIG. 6 illustrates the components of the bio-sensor stimulation electrode embodiment. In this embodiment, a signal is received at (6.1), and is converted to power, (5.1), which controls the constant current stimulator (5.13). A low power consumption is required to power a single channel (between 2 and 5 watts). A constant current (mA) stimulator (5.13) provides a stimulus via a biphasic constant current (mA), (5.9), to the subject through the proximal edge of the electrodes, (5.10) and (5.11). The intensity of the stimulus may be modified at (5.12), and duration of the stimulus controlled at (5.14) having amplifier (5.77) and control electronics (5.5).

FIG. 7. represents the components of the EMG bio-sensor electrode embodiment. The EMG signal is received via electrodes (5.10) and (5.11). Once amplified, (5.9), and filtered, (5.3), the EMG signal is allowed to free run into buffer, (5.16), then into storage buffers (5.15). After processing, (5.4), the EMG signal is continuously converted to a digital signal (5.6), and transmitted via the LED and displayed at computer screen, (15).

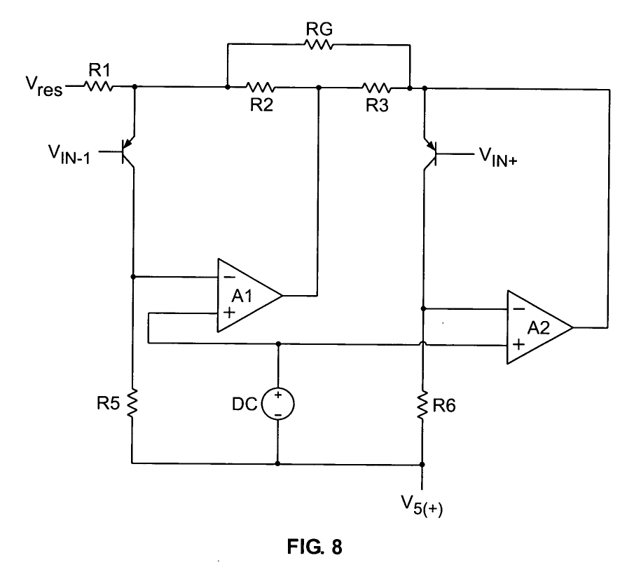

FIG. 8 is described below.

The invention is based on newly designed microelectronics encompassing analog, mixed-signal and digital IC design, CMOS, Bipolar, and BiCMOS technologies and processes, and having advanced mixed-signal design and layout.

The system is controlled by a custom designed software, based on a Tiny OS™ operating system, a sensor-based technology for remote biological monitoring applications. The software implements wireless data acquisition, signal processing, signal transmission, signal reception, data storage, display and real-time assessment incorporating custom software for performing real-time comparison, assessment, monitoring, and storage. Tiny OS components have been written to implement wireless data acquisition and transmission access control with MAC-media-access-control™ protocols for the bio-sensor.

The bio-sensor operates on a standard Tiny OS™ component to receive and display data from a USB connection. The modified data acquisition component implements a single channel acquisition and accumulation algorithm to maximize data-throughput, with high data resolution. A Java™-based program has been written to display the received waveforms on a PC personal computer. This program acquires data from the USB port and displays them as reconstructed waveforms. Signal reconstruction is performed by padding the original signal and passing it through as 8^{th} order Chebyshev filter. The Tiny OS platform has been designed to operate on a component-based run-time environment that specifically provides support for systems with a minimal amount of hardware.

Each bio-sensor in the network has communication, I/O, and processing capabilities, allowing each to act as data-router, sensor interface and control point simultaneously allowing for networking of multiple sensors. The Tiny OS enabled bio-sensor platform provides a set of intimately interconnected “components” to facilitate cross-layer optimizations, which grants high-level applications with direct and efficient control over low-level hardware. This allows the customized software to implement application specific high-level networking and data communication protocols, and to control low-level hardware such as photocouplers for optimal performance. The customized software has developed a custom network and communication protocols specifically for the bio-sensors.

The bio-sensor combines data acquisition, signal processing, signal averaging, power management and communication capabilities on the recording bio-sensor, data acquisition, signal processing, power management and communication capabilities on the sEMG bio-sensor, and signal processing, stimulus control, power management and communication capabilities on the stimulus bio-sensor.

Featuring signal acquisition, data processing and communication capabilities, the bio-sensor is approximately 2.5 cm in diameter, and approximately 12 mm thick, but those skilled in the art will appreciate that the size of the bio-sensor may alter to accommodate different technical specifications or needs. In one embodiment, a larger recording surface area is used. The bio-sensor is powered by a near infrared (ir) light source: an ir-modulated light beam is directed toward the exposed light collector atop the sensor, the collected light is focused onto a silicon PIN photodiode, and the photodiode converts light into the current needed to operate the sensors electronic components. Power for the bio-sensor is in the order of microwatts (μW). The architecture of the bio-sensor consists of variations of data acquisition; data processing; optical communications; power management; I/O expansion; and secondary storage.

The bio-sensor comprises user-programmable data modulation frequencies, a fast processor and high data throughput. The bio-sensor is powered via an ir-transceiver station that connects via a PC-USB interface to a personal computer. The transceiver station comprises transmitter circuits, controlling a pulsed light emitter, providing a light source that is intensity-modulated to match a light receiver. To produce the highest possible light pulse intensity, a low-duty cycle drive is employed, by driving the LED (complex semiconductors that convert an electrical current into light) with high peak currents with the shortest possible pulse width and with the lowest practical pulse repetition rate. For the sake of efficiency, the LED is driven with a low-loss transistor, and power field effect transistors (FET). Given the long-range application, the LED must be bent into a tight light beam to insure a detectable amount of light reaches the distant receiver. Therefore a wide divergence angle specification is used in calculating lens placement. Multiple light sources or wide area light transmitters may be employed. Angle diversity for non-directed wireless infrared communication, or multi-beam transmitters, with signal splitters, and imaging diversity receiver's principles, may be incorporated in the design.

The infrared LED, a GaAlAs (gallium-aluminum-arsenic) ir-LED, produces light that matches silicon PIN detector response curves. They are packaged in molded plastic assemblies, with small 3/16 lenses. The position of the chip within the package determines the divergence of the exiting light. When used with large lens, it can be used for longer range distances. It will further provide, receiver circuits, which will extract data information that has been placed in the modulated light carrier by the bio-sensor transmitter and restores the data to its original form. Circuits collect the modulated light from the transmitter with a plastic lens and focus it onto a silicon PIN photodiode, light detectors (PIN)-stray light filters (in reversed biased-mode, it becomes a diode that leaks current in response to light striking it, the current is directly proportional to the incident light power level-stray light filters can be placed between the lens and the photodiode), current-to-voltage converter (converts the current from the PIN to voltage-high impedance detector, resistor feedback, inductor feedback, limited Q), post-signal amplifier (signal filter, noise reduction), signal pulse discriminator (comparator) and decoding circuits (sensor coding, display).

The heart of the sensor is a microprocessor based on an Atmel ATmega 128L™ that operates at 7.372 mHz, and contains 128 kB of on-board flash memory (for storing the program that operates the bio-sensor) as well as 4 kB EEPROM (for bio-sensor configuration), 4 kB SRAM (for program memory) and a 16 bit analog. Secondary data storage is handled by an Atmel AT45 DB041 serial flash memory array. The 512-kB capacity of this memory array enables the bio-sensor to locally store or relay over 100,000 measurements to the system's USB port. The infrared transceiver station is able to emit and receive from up to sixteen individual bio-sensors.

The recording averaging bio-sensor has custom micro-circuits and micro-controllers, system-on-a-chip (SOC) for ir-light transmission LED and reception PD, signal acquisition. The recording bio-sensor receives a modulated light transmission to power on. The bio-signal between the two disc electrodes is pre-amplified (differential op-amp) with DC correction. Signal processing is as follows: (i) filter through low-pass/high-pass filters; (ii) the filtered signal will have a Gain applied to the analog signal; (iii) the signal is recorded in windows of 30, 50 or 100 ms, and is then averaged 128 times. Signal averaging follows. The summated averaged analog signal is then converted to a digital representation. The signal is converted by an analog digital converter (ADC): the signal is then conditioned to assess the peak linear aggression of the summated signal to assign a digital latency. The assigned digital latency is modulated for light transmission to the receiver. The signal is transmitted via an LED that converts current into light. Individual light transmissions are sensor-specific coded which are then decoded by the receiver software.

An sEMG (spontaneous electromyographic activity) bio-sensor has custom micro-circuits and micro-controllers, SOC for ir-light transmission LED and reception PD. Signal Acquisition is as follows: the signal is recorded from two percutaneously introduced needle (12 mm/27 g) electrodes. The bio-signal between the two needles is pre-amplified (via the differential-op-amp) then processed. Signal processing comprises: passing to low-pass and high pass and EMG notch filters; gain is added to the signal; the signal is recorded in a window of 100 ms free run; the accumulated signal is then buffered to allow a new window to be recorded, the accumulated signal is digitally converted via the ADC, and modulated for light transmission to the receiver.

A stimulation bio-sensor will have custom micro-circuits and micro-controllers, SOC, ir-light transmission and reception. Signal reception will power on the sensor. The stimulus circuit provides transcutaneous stimulation in software-controlled intensities of 0.5 mA to 10 mA, and in software controlled durations of 0.5 ms to 2.56 ms. Stimulus is delivered by two 8 mm gold disc electrodes attached to the subject's skin by a layer of medical grade adhesive.

In the clinical setting, the Light System Configuration (LSC) between the bio-sensors and transceiver station (TS) uses a diffuse reflective configuration, with beam splitting to saturate an entire room. Intraoperative monitoring employs the use of diffuse reflective configuration with NeuroNet™, a custom apparel for limbs and trunk, having designed apertures for use with a particular electrode montage. The NeuroNet system has infra-red light diffused through the fibers of the apparel to reflect the signal when the subject is in the operating room under covers, with ir-light source reflectors for lowers and ir-light source reflective covers for uppers.

The bio-sensor operates in low power, no power, and power on power off situations. Recording/averaging bio-sensors are in a low power status throughout the monitoring/testing process. Stimulus bio-sensors operate in a power on (individual site being stimulated) then power off, and are networked to the next stimulation site, per software stimulation protocols. sEMG bio-sensors are power on for continuous recording from the site throughout the monitoring/testing process.

Since the wireless bio-sensor recording system requires continuous high data-throughput, cross-layer optimizations are tailored for maximum data-throughput achievable by the hardware. In addition, accurate signal reconstruction requires very accurate sampling intervals, therefore very precise timers are used that are immune to interrupt conflicts. Data-access protocols are implemented that set the conditions and methods by which each bio-sensor will send and receive data.

In another aspect, the bio-sensor recording system consists of three major components:
(i) wireless bio-sensors;
(ii) base transceiver station; and
(iii) software enabled personal computer, the software comprising controlling the bio-sensors, controlling the transceiver station, reading the data from the USB port, displaying the data and assessing the data.
   The wireless bio-sensors acquire, and digitally encode packages and transmit a single channel of signal over an ir-band. The bio-sensor consists of an electronic interfaced with custom designed circuits and micro-controllers, powered by photocoupler technology, and having an exposed ir-transmitter/receiver. The base transceiver station (TS) has ir-transmitters and receivers, and is powered by the PC USB port. The TS can control up to 16 channels of bio-sensor data, sending data calls to the USB port of the PC. The custom software enables a personal computer to acquire the signal from the USB port, and uses digital signal reconstruction algorithms to display the original signal.

The bio-sensor carries a 16 bit analog digital converter (ADC) capable of acquiring and digitizing single ended analog signals referenced to a photocoupler power source. In one embodiment, bio-signals in the μV to mV (microvolt to millivolt) range are sensed by a pair of 8 mm gold electrodes (encased in an electrolyte gel) to correct the DC bias. The analog circuit must DC-reference, amplify, and convert the signal from differential to single-ended signal. To make this available across the dynamic range, the DC-reference point must be set to half the power voltage, while the gain is large enough to display baseline activity with the given signal resolution (16-bits which yields 510 data points) while avoiding saturation.

The neural amplifier is an Analog Devices AD627™ instrumentation amplifier. A data-acquisition, medical grade instrumentation amplifier is a closed-loop gain block that has differential input and output that is single-ended with respect to a reference. The input impedance of the input terminals is normally balanced and has very high values of ˜10 GΩ (gigaohms). The input bias currents are typically low, ˜10 μA (microamps), output impedance is generally on the order of a few mΩ (milliohms) at low frequencies. The gain of the instrument amplifier is determined by an internal resistive network that is isolated from its input terminals. The external resistor is incorporated as part of the resistive network that determines the gain, allowing the user to set the gain by specifying a certain external resistor value.

The AD627™ is a monolithic instrumentation amplifier that embodies a modification of a two-op-amp instrumentation amplifier. If we initially neglect the gain resistor R_{G9} the feedback loop comprised of R₅, V₁, and A₁, force a constant DC current (equal to V₁/R₅) through Q₁. This causes Vᵢₙ₁ to appear at the emitter of Q₁, thus resulting in a voltage equal to (1+R₂/R₁)V_{in 1} to appear at the output of A₁. Similarly, the feedback loop comprised of R₆, V₁, and A₂, force a constant DC current (equal to V₁/R₆) through Q₂, which causes Vᵢₙ₂ to appear at the emitter of Q₂. If R₁=R₄=100 kΩ, and R₂=R₃=25 kΩ, then the small-signal gain from the output of A1 to the output terminal will be 4, which results in a gain of 4×(1.25)=5 from Vᵢₙ₁ to Vₒᵤₜ. The gain experienced by the signal on the emitter of Q₂ (Vᵢₙ₂) is also equal to 5 when both loops are balanced, thus making the gain from the inverting and non-inverting terminals equal. The differential mode gain is thus (1+R₄/R₃), and by adding the external gain resistor R_{G9}, the gain will increase by (R₄/R₁)/R_{G}.

FIG. 8 shows a diagram for the instrumentation differential OpAmp amplifier (A1/A2) designed to increase the out voltage while addressing the removal of the bias of the DC current at the electrode sites, balancing each amplifier, getting the same gain from inverting and non-inverting terminals, and adding an external gain resistor, R_{G}, to increase the overall gain out.

The 16 bit analog to digital converter that is built into the bio-sensor is capable of digitizing analog signals that lie between ground and the power voltage. For neural signals sampled at a given rate, higher data resolution requires a greater bandwidth (or data throughput). The ADC must provide 16-bit resolution with available sampling rate of 200 kHz down to 0.2 Hz with a linearity error of ±2 LSB. Since the neural signals are recorded differentially, the output signal must be single-ended and referenced to the mid-point of the available dynamic range to facilitate positive and negative swings of the output. Therefore, the DC reference point must be set at half the power voltage. The gain of the preamplifier also must be set to be large enough to make the most of the available 16-bit resolution.

The Tiny OS applications are written in neSC™. NeSC™ is a language that has recently been developed for programming structured component-based applications Intended for embedded systems such as sensor networks, Tiny-OS is composed of components that implement and use interfaces that execute commands (which progress down the software hierarchy) and handle events (which progress up the software hierarchy). An interface is a generic declaration of commands and events which are implemented by the interface provider. The two types of components used in nesC are modules and configurations. Modules provide application code, implementing one or more interface. Configurations connect components that provide interfaces to those that use them, thus assembling (or wiring) components together. Custom Tiny-OS components have been developed to implement data-acquisition, data-transmission, data-reception, in addition to modified media-access control (MAC™) protocols to maximize the available bandwidth capabilities of the hardware. The MAC layer is of critical concern when optimizing a system built on multiple bio-sensors. A dedicated data-collection paradigm allows for simplification of communication protocols that permit communicative liberation. Software components are written to implement data acquisition and wireless MAC protocols for the bio-sensor transmitter. The bio-sensor receiver operates on standard Tiny OS components to receive data and send them to the USB port of the PC. The bio-sensor operates on a custom data acquisition component. The custom software is required for enabling a PC to interpret and display the data that streams into its USB port via MIB510. The Tiny OS 1.1 release includes two Java applications: SerialForwarder™ and Oscilloscope™, which forward the data from the serial port to a TCP/IP port, and display data received from the TCP/IP port, respectively. The SerialForwarder™ application used was modified to allow for USB port configuration. The Oscilloscope™ application was also modified, with digital signal reconstruction techniques to synthesize the original to waveform from the sampled data points. An accurate method of reconstructing a sampled signal uses a frequency domain representation (Fourier transformation) of the sampled signal to arrive at a close representation of the original signal, as long as the signal was sampled at double its theorem, a signal with frequency components ranging from DC to 125 Hz (or half the sampling frequency) can theoretically be fully reconstructed. To maximize the useful signal bandwidth, given the available data throughput, a signal reconstruction algorithm was developed using MATLAB™ software and signal conditioning software.

Far-field potentials are generated by movement of a charge causing a front of depolarization and repolarization. For example, the posterial tibial nerve is stimulated and a recording is produced at the site of the bio-sensor, which could placed over any far field volume conductor such as, the posterior spinal column, the cerebral cortex, or the lumbar sacral spine, where the window in which the recording over the bio-sensor is being made, is time-locked to the delivery of the stimulus. For example, if at time t=zero, a stimulus is delivered, a recording is captured over the lumbar sacral spine in about 40 seconds. Typical recording time windows are shown in Table 2.

TABLE 2

| | | Recording |
|---|---|---|
| | | Window |
| Stimulating | Recording | (msec) |
| Posterior Tibial | Subcortical | 100 |
| | Posterior Cervical | |
| | spine | |
| Median | Subcortical | 50 |
| | Posterior Cervical | |
| | spine | |
| | Muscle EMG | 100 |
| Near nerve | Neurogenic Evoked | 30 |
| Cervical Spine | Potential | |
| Cortex | | |
| Cervical Spine | Motor Evoked | 30 |
| Cortex | Potential (MEP) | |
| | ECG | 30 |
| | EEG | 15-30 |

From the point of stimulation of the lower extremities to recording signal over the posterior cervical spine or cerebral cortex, the recording window of a lower extremity nerve, for example, the posterial tibial nerve, or of a dermatome, is 100 milliseconds (msec). The recording window from the point of stimulation at the upper extremities (the median nerve) to the cervical spine or cerebral cortex, is 50 ms. In compound action muscle potentials in which recording is being made from the muscle, the time-window is 30 ms. In neurogenic evoked potentials (in which recording takes place at a nerve, and stimulation may be of any segment proximal to where a signal is being recording from), the time window is 30 ms. For EMG, the time window is 100 ms.

One embodiment of the wireless electrode comprises pattern-recognition algorithms for data compression wireless transmission, modulation and multiplexing schemes and circuits CM, FM and sigma delta for signal transmission. Such an approach minimizes 50 Hz power line interference, either via impedance matching or impedance transformation. The contemplated wireless electrode constitutes a universally safer power source, the power source being contained, and are not connected to a mains power source.

The wireless electrode comprises pattern-recognition algorithms for data compression wireless transmission, modulation and multiplexing schemes and circuits CM, FM and sigma delta for signal transmission. Such an approach minimizes 50 Hz power line interference, either via impedance matching or impedance transformation. The contemplated wireless electrode constitutes a universally safer power source, the power source being contained, and are not connected to a main power source.

### EXAMPLE 1

### Bio-Sensor Averaging Recording Electrode

In this example, bio-sensor averaging recording electrodes designed for either upper or for lower extremity monitoring/testing are placed over or near a far-field potential generating site for acquisition and amplification of such electrophysiological potentials. The electrical activity recorded at the recording site is processed at the site of recording wherein the recorded signal is amplified, filtered, averaged, summated, digitally converted and transmitted to the computer for display/assessment.

This bio-sensor uses bandwidth filtering of high pass: 2 Hz, and low pass: 100 Hz with Gain 20 μV, activated in series with the serial time-locked stimulation protocol. Bio-sensors are placed over the bilateral brachial plexus and posterior cervical spine. The stimulation site is over the C6 distribution, distal to the recording site. The averaging recording electrodes are activated in series using a serial time-locked stimulation protocol.

A differential OpAmp receives the input from a pair of cutaneous recording electrodes (8 mm disc Ag-AgCl) placed over the posterior cervical spine, where fast and weak bio-signal in the 0.02 Hz to several thousand Hz is occurring, in the 10-20 μV range. These fast occurring, low amplitude signals are picked up by the electrodes and are amplified by the differential (Input 1+Input 2−) OpAmp. The signal amplifying electronics has low noise input (not exceeding 10 μV) and a good DC rejection of randomly occurring slow potentials (by generating high resistance in parallel to the capacitor in the feedback loop) with capacitors and transistors that improves noise performance. Since the sensors are recording and processing signal at the site of occurrence, low noise and high signal to noise ratios (SNR) are at unprecedented levels. Signal filtering is accomplished with band pass filtering, the range of the filters being: High Pass of 0.02 Hz to 10 Hz and Low Pass of 50 Hz to 5 KHz. The low amplitude signals are enhanced by applying a gain to the signal, adjustable from 5 μV to 100 mV. Signal processing electronics includes signal averaging with summations up to 128 sweeps, producing a sampling rate of 4-20 KHz, with 128 samples in 16 bit resolution, in recording windows of 50 and 100 ms which will be time-locked to the delivering of a stimulus provided by a bio-stimulating electrode selectively positioned distal to the recording site over cutaneously distributed nerve roots or mixed nerve sites. The summated averaged signal is converted to a digital representation by an analog-to-digital A-D converter. The digital signal is transmitted from the bio-sensor, via the light emitting diode (LED) to the wireless receiver, the photodiode, for signal display and assessment. The operational electronics and signal transmission is optically powered with a near infrared light source.

The following lists the electronics necessary for acquisition and amplification of electrophysiological potentials, incorporated into a transmitter:

wireless power/data reception/transmission.

a compact photo coupler-like system for digital data transmission.

optically powered near infrared light transmitter photodiode (PD) source.

LED light emitting diode for transmission and powering the sensors.

specific sensor detection by using optical filter labeling.

For a single channel device:

1. Differential (OpAmp). The amplifier for biosignal recording has low noise input and good DC rejection. Low noise can be achieved either by having wide input PMOS and large load transistors or by using chopper modulated technique. OpAmp is designed as a two-stage voltage amplifier.

2. Noise analysis. The following equation is used to calculate Low Noise (numerator n).

n=16KT/3 1\3 1\gm2/0{gm0+gm8}+gm15+gm13/gm2/13(ro1∥ro8)2V2ni,thermal

Stage load over the transistors is spread out: Gm0, gm8, gm13, gm15 transconductances of input PMOS's staged load transistors, input PMOS should be wide and input large.

3. Capacitors are added between first and second stage to limit the bandwidth of the OpAmp. Transistors may be added to minimize transient voltages slew-rate limiting, and help with lower common mode gain and improve noise performance. OpAmp has fully differential configuration, with capacitively-coupled inputs

4. DC rejection by generating high resistances in parallel to the capacitor in the feedback loop.

5. 8 mm disc electrodes gold plated, Ag, Ag AgCl, or tin.

6. High SNR (signal to noise ratio).

7. Amplification of signals in the 10-20 μV range.

8. Bio-signal fast and weak, 0.02 Hz to several thousand Hz.

9. Frequency response for transmission to the input signal 0.02 Hz to 5 KHz (−3 dB).

10. Low input noise not exceeding 10 μV.

11. Differential input: input (1)+ input (2)−.

12. Signal averaging 128 sweeps.

13. 50 ms upper, 100 ms lower, (30 ms-NEP, MEP) recording windows.

14. Sampling 4-20 KHz @ 50 ms/100 ms, 128 samples 16 bit resolution

15. Normal bandwidth filtering:

High Pass 0.02 Hz to 10 Hz
Low Pass 50 Hz to 5 KHz
Adjustable Gain: 5 μV, 10, 20, 50, 100, 500 μV-1 mV, 2, 5, 10, 20, 50, 100 mV

Notch filter 50/60 Hz is optional.

Data Acquisition Specifications

Analog Inputs

| Connection type: | Gold disc electrodes (8 mm pair) | |
|---|---|---|
| Input channels: | 1 | |
| Input configuration: | differential | |
| Amplification range: | Range | Resolution |

| | ±10 | V | 312.5 | μV |
|---|---|---|---|---|
| | ±5 | V | 156.25 | μV |
| | ±2 | V | 62.5 | μV |
| | ±1 | V | 31.25 | μV |
| | ±0.5 | V | 15.625 | μV |
| | ±0.2 | V | 6.25 | μV |
| | ±0.1 | V | 3.125 | μV |
| | ±50 | mV | 1.56 | μV |
| | ±20 | mV | 625 | nV |
| | ±10 | mV | 312.5 | nV |
| | ±5 | mV | 156.25 | nV |
| | ±2 | mv | 62.5 | nV |
| Maximum input voltage: | ±15 V | | | |
| Input impedance: | ˜1 M Ω\| \| 47 pF @DC | | | |
| Low-pass filtering | 25 kHz fixed 2^{nd} order (further filtering via | | | |
| | software) | | | |
| Frequency response (−3dB) | 25 kHz @ ± 10 V full scale, all ranges | | | |
| CMRR (differential): | 96 dB @ 50 Hz (typical) | | | |
| Input noise: | <2.4 μV rms referred to input | | | |
| Sampling | | | | |
| ADO resolution: | 16 bit | | | |
| Linearity error: | ±2 LSB (from 0 to 70° C.) | | | |
| Maximum sampling rates: | 200 kHz | | | |
| Available sampling rates: | 200 kHz down to 0.2 Hz | | | |
| Output Amplifier | | | | |
| Output configuration: | differential (complementary) | | | |
| Output resolution: | 16 bits | | | |
| Maximum output current: | 100 mA (max) | | | |
| Output impedance: | 0.4 Ω typical | | | |
| Slew rate: | 6.v/μs | | | |
| Settling time: | 2 μs | | | |
| Linearity error: | 1 LSB (from 0 to 70° C.) | | | |
| Output range: | 200 mV to ±10 V (software-selectable) | | | |
| | Range | | Resolution | |
| | 10 | V | 312.5 | μV |
| | 5 | V | 156.25 | μV |
| | 1 | V | 31.25 | μV |
| | 500 | mV | 15.625 | μV |
| | 200 | mV | 6.25 | μV |

| Data Communication | max 480 Mb/sec transfer | |
|---|---|---|
| External Tripper | | |
| Trigger mode: | TTL level (isolated) or contact closure (non- | |
| | isolated) software selectable | |
| Trigger threshold: | +1.2 V ± 0.5 V (TTL compatible) | |
| Hysteresis: | >0.5 V (turns off at 2.8 V ± 0.25 V) | |
| Input Load: | 1 TTL load | |
| Maximum input voltage: | ±12 V | |
| Minimum event time: | 5 μs | |
| Operating temperature: | 0 to 35° C., 0 to 99% humidity (non- | |
| | condensing) | |
| Bio-Sensor Amp Specifications | | |
| Input | | |
| Connection type: | 2 gold disc 8 mm electrodes | |
| Input configuration: | isolated differential | |
| Input impedance; | 200 M Ω differential | |
| Safety: | Approved to IEC601-1 BF9 body protection—or | |
| | 1EC601-1 CF(cardiac protection) standard | |
| Isolation: | 400 V ᵣₘₛ (50 Hz for 1 minute) | |
| Amplification ranges: | 5 μV to ±100 mV full scale in 14 steps | |
| | ±100 | mV |
| | ±50 | mV |
| | ±20 | mV |
| | ±10 | mV |
| | ±5 | mV |
| | ±2 | mV |
| | ±1 | mV |
| | ±500 | μV |
| | ±200 | μV |
| | ±100 | μV |
| | ±50 | μV |
| | ±20 | μV |
| | ±10 | μV |
| | ±5 | μV |

| Gain accuracy: | ±1.5% all ranges | |
|---|---|---|
| Non-linearity: | <0.1% within range | |
| Noise at various band widths: | 1 Hz to 5 Hz | <1.3 μV ᵣₘₛ |
| | | (<8 μVp-p) |
| | 0.3 Hz to 1k Hz | <0.6 μV ᵣₘₛ |
| | 0.1 Hz to 100 Hz | <0.35 μV ᵣₘₛ |
| | | (@ 200 samples/second) |

| | |
|---|---|
| IMRR (isolation): | >130 dB (50-100 Hz) |
| CMRR (common mode): | >76 dB (10 Hz to 1 kHz) |
| Input leakage current: | <3 μAᵣₘₛ @ 240 V, 50 Hz |
| | <2 μAᵣₘₛ @ 120 V, 60 Hz |
| Filtering | |
| Low-pass filtering: | Fourth-order Bessel filter, ±3% accuracy. |
| | Frequencies software-selectable. Standard |
| | 50, 100, 200, 500, 1000 & 5000 Hz (@-3 dB) |
| | EEG mode: 3, 10, 30, 60 and 120 Hz |
| High-pass filtering: | First-order filter ±0.25% accuracy. |
| | Frequencies software-selectable, Standard |
| | 0.1, 0.3, 1,3, and 10 Hz (@-3 dB) |
| | EEG mode: 0.03, 0.1, 0.3, and 1 seconds |
| Notch filtering: | Second-order filter, −32 dB attenuation; |
| | 50 or 60 Hz frequency |
| Output | |
| Analog signal: | 2.0 V standard |
| Communications | rate of ˜50 Kbits/s. |
| Operating temperature range: | 0 to 35° C., 0 to 90% humidity |
| | (non-condensing) |

### EXAMPLE 2

### Free Run Needle EMG Bio-Sensor Electrode

The free-run electromyographic activity electrode bio-sensor used in this example is a single channel device, housing the internal electronics necessary for acquisition, processing and transmission of spontaneously occurring muscle potentials. The bio-sensor free-run needle EMG electrodes are placed over the musculature that is to be evaluated, insertion of the needles is accomplished by pressing the lateral insertion tabs on the sensor, spontaneous free run EMG activity for the muscle is amplified recorded and transmitted to the computer, through the optically powered near infrared light transmitter photodiode unit for display and assessment. All power and data reception/transmission are accomplished with infrared light source, using photo coupling technology.

In this example, the differential OpAmp receives input from a pair of 13 mm/27 gauge needle electrodes placed percutaneously in the bicep musculature, where fast and weak muscle activity occurring in the few hundred to several thousand Hz range are occurring, and in the 20 μV to several mV range. This low amplitude fast occurring signal is picked up by the needle electrodes and is amplified by a low noise, high DC rejection OpAmp, with capacitors and resistors to lower noise and improve signal to noise ratios (SNR) The amplified signal is filtered with band-pass filters, High Pass 2 KHz. Low Pass 10 Hz and is enhanced by applying 20 μV of gain to the signal. The rapidly occurring enhanced signal is buffered, converted to digital representations, via an A-D converter, and is continuously transmitted in real time. The processed signal is transmitted via the light emitting diode (LED) to the wireless receiver photo diode for signal display and assessment. The operational electronics and signal transmission are optically powered with a near infrared light source.

The following describes the electronics necessary for free run needle EMG, incorporated into a transmitter:

single channel device

differential OpAmp, isolated

potentiometer

constant current stimulus (mA)

gain 20,000 mV

13 mm 27 gauge needle electrodes

Specifications for the needle bio-sensor are as above.

### EXAMPLE 3

### Bio-Sensor Stimulating Electrode

For wireless bio-stimulation, the electrodes are placed on the skin at pre-determined stimulation sites, over dermatomal nerve root distributions or over peripheral mixed nerve distributions. Activation and control of the electrode is software controlled by the computer through the “Phosphor” photodiode unit, the optically powered near infrared light transmitter. Surface stimulation is time-locked.

In this example, each bio-stimulation electrode bio-sensor comprises a single channel device, housing the internal electronics necessary for controlling and delivering a constant current stimulus. The micro constant current stimulator receives activation input via a light receiver to deliver a constant current biphasic trains of pulses in mA intensities of 0.10 μA to 10.0 mA, controlled in durations of 0.01 ms to 2.56 ms and delivered by two cutaneously oriented 8 mm disc AG-AgCl electrodes, individually designated as either an anode or cathode. The operational electronics and signal reception are optically powered with a near infrared light source.

The bio-sensors, systems, apparatus and methods herein provide distinct advantages over prior equipment. Thus, reference herein to specific details of the illustrated or other preferred embodiments is by way of example and not intended to limit the scope of the appended claims. It will be apparent to those skilled in the art that modifications of the basic illustrated embodiments may be made without departing from the spirit and scope of the invention as recited by the claims.

The following describes the necessary electronics and specifications for wireless bio-stimulation, incorporated into a transmitter:—

Single channel device.
8 mm disc electrodes.
Anode/cathode.
Deliver 2.8 mA constant current =/− 5% accuracy.
1.56 ms duration with applicable time locked delays of 19 ms, 23 ms,
24 ms, 43 ms, 44 ms.
Biphasic stimulation Functional Electric Stimulation (FES) charge
balancing over trains of pulses.
Rectangular biphasic stimulation pulses (2.8 mA 1.56 ms duration).
Specifications:

| | |
|---|---|
| Connection type: | Gold disc electrodes (8 mm pair) |
| Output configuration | Constant-current stimulator with hardware limited |
| | repetition rate, with following discharge clamp |
| Output waveform: | Rectangular, monophasic pulses with software- |
| | set amplitude and duration |
| Safety: | Approved to IEC601-1 BF (body protection) |
| | standard |
| Isolation rating: | 4000 V AC ᵣₘₛ for 1 minute |
| Safety indicators: | A single multi-color indicator displays the |
| | isolated stimulator status. A green flash indicates |
| | delivery of a valid stimulus. A yellow flash |
| | indicates an out-of-compliance condition (OOC). |
| Safety switch: | Isolating On-off switch flicks down to disconnect |
| | quickly |
| Compliance voltage: | 100 V fixed |
| Current ranges: | 100 μA. 1 mA, or 10 mA full scale |
| Current rise time: | <1 μsec (1 kΩ load @ 10 mA) |
| | 25 μsec (100 kΩ load @ 0.5 mA) |
| Current fall time: | <1 μsec (1 kΩ load @ 10 mA) |
| | 25 μsec (100 kΩ load @ 0.5 mA) |
| Operating duty cycle: | up to 20% |
| Resolution: | 1% of full scale (1 μA, 10 μA, or 100 μA) |
| Leakage current: | <200 nA p-p |
| Differential output | <1 μA p-p |
| noise: | |
| Power source: | Isolated and high voltage circuitry derives power |
| | from the IR diode,light source, isolation by an |
| | isolation transformer |
| Pulse duration range: | 0.01 ms (10 μs) to 2.56 ms in 0.01 ms (10 μs) |
| | steps |
| Duration accuracy: | 0.01% +5/−0 μs |
| Repetition rate: | 2 pulses per minute (0.0333 Hz), up to 200 Hz. 1 |
| | pulse per minute (0.017 Hz), up to 200 Hz with |
| | enhanced software |
| Repetition accuracy: | 0.1% |
| Current rise delay: | 12-22 μs (variable) |
| Control: | Long range, interface communication rate of ˜ |
| | 50 kbits/s. LED controller provides power and |
| | control |
| Operating temperature | 0 to 35° C., 0 to 90% humidity (non-condensing) |
| range: | |

What is claimed is:

1. A wireless bipolar bio-sensor for attaching to the body of a subject for recording a biopotential signal, the bio-sensor comprising:
a) a pair of electrodes capable of recording a biopotential signal from a subject;
b) a differential amplifier in contact with the electrodes and capable of generating an amplified differential signal from the signal recorded between the electrodes;
c) a miniaturized system-on-a-chip (SOC) attachment in contact with the differential amplifier, configured to process the signal received from the amplifier; and
d) an infra red light transmitter/receiver connected to the SOC attachment and capable of receiving optical power from a remote ir-light source transceiver, and of transmitting the signal thereto.

2. The bio-sensor of claim 1, wherein bio-sensor is optically powered by a remote ir-light source transceiver capable of transmitting optical power to the sensor and receiving a signal therefrom.

3. The bio-sensor of claim 1, wherein the electrodes are discs.

4. The bio-sensor of claim 1, wherein the electrodes are silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof.

5. The bio-sensor of claim 1, wherein the signal is a measurement of the subject's spontaneous activity.

6. The bio-sensor of claim 5, wherein the signal is an electromyographic signal, an electrocardiographic signal or an electroencephalographic signal.

7. The bio-sensor of claim 5, wherein the signal is a measurement of the subject's response to a pathology experienced by the subject.

8. The bio-sensor of claim 7, wherein the pathology is a result of a trauma, a circulatory change, a degenerative change, a metabolic change, an infection, a chemical insult, radiation, or a neoplastic change.

9. The bio-sensor of claim 7, wherein the pathology is the result of a surgical intervention.

10. The bio-sensor of claim 1, wherein the signal is elicited from the subject in response to an applied stimulus.

11. The bio-sensor of claim 10, wherein the response is time-locked to the stimulus.

12. The bio-sensor of claim 10, wherein the signal is a somatosensory evoked potential, a dermatomal somatosensory evoked potential, a motor evoked potential or a nerve conduction potential.

13. The bio-sensor of claim 10, wherein the applied stimulus is electrical, sonar, mechanical, tactile or optical.

14. The bio-sensor of claim 11, wherein the signal results from a change in the subject's response to the applied signal as a result of a pathology experienced by the subject.

15. The bio-sensor of claim 14, wherein the pathology is a result of a trauma, a circulatory change, a degenerative change, a metabolic change, an infection, a chemical insult, radiation, or a neoplastic change.

16. The bio-sensor of claim 14, wherein the pathology is the result of a surgical intervention.

17. The bio-sensor of claim 11, wherein the SOC attachment is configured to integrate the following: signal acquisition; filtering the signal; averaging the signal; summating the averaged signal; converting the signal to a digital signal; signal conditioning to assign a digital latency value; and transmitting the digital signal to a remote receiver.

18. The bio-sensor of claim 17, wherein the pair of electrodes is housed in a first layer having on its distal surface an adhesive area for cutaneous or percutaneous conductive attachment to the subject's musculature, the pair of electrodes being transferred to an electrode substrate material proximally in contact with a second unexposed layer comprising the differential amplifier, the SOC attachment and the infra red light transmitter/receiver, the second layer being covered by a third exposed layer comprising an insulating material and extending to the circumferential borders of the first layer, the third layer having a transparent portion for transmitting and receiving power.

19. The bio-sensor of claim 17, wherein the electrodes are silver-silver chloride.

20. The bio-sensor of claim 10, wherein the electrodes are needle electrodes.

21. The bio-sensor of claim 20, wherein the needle electrodes are in-housed percutaneous needles for percutaneous attachment to the subject's musculature.

22. The bio-sensor of claim 20, wherein the proximal ends of the needles are attached to the SOC attachment and embedded in an electrode substrate material.

23. The bio-sensor of claim 20, wherein the bio-sensor allows for adaptation of the needles.

24. The bio-sensor of claim 20, wherein the SOC attachment is configured to integrate the following: signal acquisition; filtering the signal; averaging the signal; summating the averaged signal; converting the signal to a digital signal; signal conditioning to assign a digital latency value; and transmitting the digital signal to a remote receiver.

25. The bio-sensor of claim 20, wherein the electrodes are silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof.

26. The bio-sensor of claim 25, wherein the electrodes are gold.

27. A bio-sensor wirelessly powered for applying an electrical stimulus to the nerve or muscle of a subject, comprising:
a) a pair of electrodes providing for delivery of an electrical stimulus to the subject's skin; and
b) a SOC attachment in contact with the electrodes, and including: a stimulus circuit providing transcutaneous stimulation to the subject via the electrodes; a receiver means for activating a constant current stimulator to deliver a stimulus; a means for controlling the duration and intensity of the stimulus; and an infra red light transmitter/receiver means connected to the SOC attachment and capable of receiving optical power from a remote ir-light source transceiver, and of transmitting a feedback signal thereto.

28. The bio-sensor of claim 27, wherein the bio-sensor is optically powered by a remote transceiver connected via a USB port to a computer and capable of transmitting optical power to the bio-sensor.

29. The bio-sensor of claim 27, wherein the stimulation is provided in software-controlled intensities.

30. The bio-sensor of claim 29, wherein the stimulation is provided in intensities of between about 0.5 mA and 10 mA.

31. The bio-sensor of claim 27, wherein the electrodes are of silver chloride, silver-silver chloride, gold, tin, a titanium base coated with iridium, platinum, or ruthenium, a precious metal or noble metal from Groups IB, IIB or VIII of the Periodic Table of the Elements, or an alloy of at least one of the metals, said alloying element being selected from the group consisting of an element from Groups IIIA, IVA, VA, VIA, VIII, IB, IIB, VIIB of the Periodic Table of the Elements, or combinations thereof.

32. The bio-sensor of claim 31, wherein the electrodes are silver-silver chloride discs.

33. The bio-sensor of claim 27, wherein the pair of electrodes is housed in a first layer having on its distal surface an adhesive area for cutaneous or percutaneous conductive attachment to the subject's musculature, the pair of electrodes being transferred to an electrode substrate material proximally in contact with a second unexposed layer comprising the differential amplifier, the SOC attachment and the infra red light transmitter/receiver, the second layer being covered by a third exposed layer comprising an insulating material and extending to the circumferential borders of the first layer, the third layer having a transparent portion for transmitting and receiving power.

34. The bio-sensor of claim 33, wherein the distal surface is a stimulating surface.

35. A neurophysiological measuring/monitoring/testing system for comparing and evaluating bio-potentials in a subject, comprising:
a) the bio-sensor of claim 1;
b) a transceiver station comprising ir-transmitters/receivers means for powering, and for data reception from, the one or a plurality of the bio-sensor; and
c) software means enabling a computer, the software comprising interacting with the bio-sensors and the transceiver station, reading the data from the USB port, displaying and assessing the data.

36. The system of claim 35, wherein the software means further comprises directing serial collection of signal data and real-time display, comparison and assessment of the collected signal data.

37. The system of claims 35 or 36, wherein a) further comprises a plurality of the said bio-sensor.

38. The system of claims 35 or 36, wherein the transceiver is powered by the computer via a USB port.

39. The system of claims 35 or 36, further comprising a software means for generating a deviation from normal warning signal via a visual, audible or electronic means.

40. The system of claims 35 or 36, further comprising a software means for providing and displaying an icon on a computer screen responsive to a command by a computer user, wherein the icon appears on the screen and prompts a user to select an option consisting of take a patient history, select a recording protocol, confirm proper electrode placement, input parameters, record a sequence, analyze data, archive data, or generate a report.

41. The system of claims 35 or 36, further comprising an apparel for the subject to wear, having apertures for guiding placement of the apertures in the stocking correlating with a specific electrode montage.

42. The system of claims 35 or 36, further comprising a plurality of the bio-sensor of claim 1.

43. The system of claims 35 or 36, further comprising one or a plurality of the bio-sensor of claim 27.

44. The system of claims 35 or 36, wherein the infrared transceiver station is able to emit and receive from up to sixteen individual bio-sensors.

45. The neurophysiological measuring/monitoring/testing system of claims 35 or 36, further comprising a computer data signal embodied in a carrier wave by a computing system and encoding a computer program for executing a computer process, the program comprising instructions for executing real-time comparison and assessment of evoked potentials, nerve conduction studies, electromyographic activity, electrocardiogram or electroencephalogram.

46. A neurophysiological measuring/monitoring/testing method, comprising:
a) attaching the bio-sensor of claim 1 to a subject at a site on a subject where an elicited signal may be recorded;
b) recording a signal in the bio-sensor elicited from a first stimulation site on the subject between the electrodes, then amplifying, filtering, averaging, summating, digitally converting and wirelessly transmitting the signal data to a remote computer; and
c) on the computer, performing wireless data acquisition from the transceiver, data storage, displaying, comparing, assessing and storing the acquired data.

47. The method of claim 46, wherein b) and c) are carried out serially in real-time.

48. The method of claim 46, wherein the elicited signal is recorded at a subcortical recording site on the subject.

49. The method of claim 46, further comprising attaching a plurality of the bio-sensor to the subject, and performing steps b)-c) with respect to each of the two or more different recording sites on the subject.

50. The method of claim 49, wherein b) and c) are carried out serially in real-time.

51. The method of claim 49, wherein the bio-sensors are attached to the subject via an apparel worn on the subject's body, having apertures for guiding placement at specific recording sites in an electrode montage.

52. The method of claim 37, wherein the elicited signal is selected from the group consisting of evoked potentials, nerve conduction studies, electromyographic activity, electrocardiogram or electroencephalogram.

53. A computer readable medium having encoded instructions for executing the method of claim 46.

54. A computer program storage medium readable by a computing system and encoding a computer program for executing a computer process, the program comprising instructions for executing the method of claim 46.

55. A method for providing a stimulus to a subject undergoing a neurological procedure to elicit a bio-potential from the subject, comprising:
a) attaching the bio-sensor of claim 27 to a subject's body where a stimulus may be provided to elicit a signal; and
b) via software means in a remote computer for interacting with the receiver means and means for controlling the duration and intensity of the stimulus, delivering a stimulus to the subject.

56. The method of claim 55, wherein the stimulus is provided to two or more different stimulus sites via two or more of the bio-sensor of claim 27.

57. The method of claim 55, wherein the two or more bio-sensors are attached to the subject via a stocking having apertures for guiding placement at specific stimulation sites in an electrode montage.
